SEQUENCE LISTING (10 sequences)

SEQ ID NO: 1 - DNA, 13 bp, synthetic construct
  source 1..13: mol_type = other DNA
attgccttatttc

SEQ ID NO: 2 - DNA, 22 bp, synthetic construct
  source 1..22: mol_type = other DNA
caacaaaatcactgatgctgga

SEQ ID NO: 3 - DNA, 21 bp, synthetic construct
  source 1..21: mol_type = other DNA
cagctatgccagcatcttgcc

SEQ ID NO: 4 - DNA, 329 bp, synthetic construct
  source 1..329: mol_type = other DNA
atattgccttatttccgattattgccttatttcgcattattgccttatttctcactattgccttatttccgatcccggggtttaaaccgatcaacaaaatcactgatgctggacgatcaacaaaatcactgatgctggacgatcaacaaaatcactgatgctggatcaccaacaaaatcactgatgctggacgatgtttaaaccctgcaggcgatcagctatgccagcatcttgcccgatcagctatgccagcatcttgcccgatcagctatgccagcatcttgcctcaccagctatgccagcatcttgcccgatcctgcaggagatct

SEQ ID NO: 5 - DNA, 22 bp, synthetic construct
  source 1..22: mol_type = other DNA
cgatcccggggtttaaaccgat

SEQ ID NO: 6 - DNA, 24 bp, synthetic construct
  source 1..24: mol_type = other DNA
cgatgtttaaaccctgcaggcgat

SEQ ID NO: 7 - DNA, 18 bp, synthetic construct
  source 1..18: mol_type = other DNA
cgatcctgcaggagatct

SEQ ID NO: 8 - DNA, 1303 bp, synthetic construct
  source 1..1303: mol_type = other DNA
cgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgatcgacggtaccgcgggccc

SEQ ID NO: 9 - DNA, 1262 bp, synthetic construct
  source 1..1262: mol_type = other DNA
acttcaggcttgtccttgagaccttgaggagagggtagacattggttctattgccccagaagcggaatttttacaacaccctactggaagaacactgatccaggaacaggaggagggagcatgaaaaaagaggacaggaaaacataggtactctatattcatctgactggcacctggcctcagataaccccaactggtgccccaggagccctattttaggatttgcaggtgcagtcttgcacaggttattttaatcggcatttccccctgaaacagcaaagcattgcaaacttaaccgtgccgcagcaataactcttaagttatggtttcggaaaaaccgtgccactgcctcagtgttcattaagcagggagactgtagaatgaattataatgatgtcccccagtttttaaaggacagtttgagggatactgaaaaggcaggagaaacaaccttaggttgcgtatgaggtgttgcagaaacggcagaggaagggtataaattttaaaaatagaaagcttctagcgttgagcaagatgcggcgttgtcggtgtttgatttctggagagggagcctacaggtagcttcctacaggtcgccacgctagttttgctgacactgaaaaagctgggaccctctaccctgcctattagagcagcagtggcccgcccaccagccccaccctgcccgccttccggagcttatctccgctcgggttccgcttctccagccagcttctggcccggccccgcctaccggcctggcccctcccaccttccggctccacccccccccccccccccaggctcgcagcctggccccacctctggacttccccaatcctctgtcgccctcgccattggtctctggctggccgcctggcccaatcgcagcacttagcgccagaatttgaaccgccgttttggtttgaatcgggcgggctgcggcgggcgagccttggcgcggaaccgcctaggcggacagtcggggctggacggggcggcgggtggcgggtggcgggcacttgggacgcgggcggcgggagtgcgggtctggtcggggcggagcgaaggccgcgggtggccgtggtcggtcctccgcggctaaggagccgagggctccgacgcgggctgcgcccggtgagcggcggccagagctaacttgcgctgactggaccagctgaggagcggcccggcggggcgactgcgagcttcaccgagaggcttctccgccctggtccgcagtcccgacggccgggcggacc

SEQ ID NO: 10 - DNA, 4991 bp, synthetic construct
  source 1..4991: mol_type = other DNA
cgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgatcgacggtaccgcgggcccaacggatccaccatggatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttggcagcggcgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggcgctagcggcagcggcaaccatgatcaagagttcgatcctcctaaagtgtatccccctgtccctgccgaaaagcggaaaccgatcagggttctcagtctcttcgatggaattgccacaggcctcttggtccttaaggacctgggcatccaagtggaccgctacatcgcctcagaagtatgtgaggacagcatcacagtcggcatggtgcgccaccaggggaaaataatgtacgtcggcgacgtcagatcagttactcagaaacatattcaggaatgggggccttttgatctcgtaattggcggcagcccctgcaatgatctgagtattgtgaaccccgcccggaaaggtttgtacgaaggtacgggcagactgttcttcgagttttacagactgttgcacgacgcacgaccaaaggagggtgacgatcgacccttcttctggctgttcgagaacgtggtcgctatgggcgtgtctgacaagcgggacatttctagattcctggagagcaatccagtgatgattgatgcaaaggaagtatccgctgcccaccgcgccagatacttctggggcaatctgcccggcatgaatcgacccttggcaagcaccgtgaatgataagttggagttgcaggagtgtctggagcatgggcggatcgcaaagtttagcaaggtgcggaccatcacgacccgaagcaattcaattaagcagggaaaggaccaacattttccagtgtttatgaacgagaaagaggacatcctgtggtgcacagaaatggagcgggttttcggattccccgtacattatactgatgtgtccaatatgtctaggttggctagacagaggctgttgggacgctcatggagtgttcctgtcatccgccacctgttcgctcctctgaaggagtatttcgcctgcgtttcatccgggaattcaaacgcaaacagcagaggcccatccttttcttccggcctggtgccacttagtctgcgcggctctcacatgggacctatggaaatatacaaaacagtgagcgcctggaagaggcagcctgtgcgggtcctgagtctgttcagaaacattgacaaggtgcttaagtcccttggatttctggagtctggcagcggttctgggggtggcacccttaagtatgtggaggacgtgacaaacgtcgtgaggagagatgtggagaaatgggggccatttgacctggtatacgggagcactcaacctctcggttcttcttgtgatcgctgcccagggtggtatatgtttcagttccatcgcatcctgcaatacgcccttccgcggcaggagagtcagcgaccattcttctggatattcatggacaatctcctgctgacagaggacgaccaagagactacgactagatttcttcagaccgaggccgttactctccaggacgttagaggtagggactaccagaatgccatgcgagtgtggagtaacatccccggactcaagtcaaagcacgcacccctgacccccaaggaagaggaatacctccaggcgcaggtgaggagccgcagtaagctcgatgctcctaaagtcgatttgttggtgaagaattgcttgctgcccctgagagagtactttaagtactttagccagaacagcttgccactgggcgcgcccggtaagcctatccctaaccctctcctcggtctcgattctacgtgaattaattaagaattcgacccagctttcttgtacaaagtggttgatatccagcacagtggcggccgctcgagtctagagggcccgcggttcgaaggtaagcctatccctaaccctctcctcggtctcgattctacgcgtaccggtgtcgactctagataaagcatcaagcttatcgcgataccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaatgatatcctcgagcccgggcgatatattgccttatttccgattattgccttatttcgcattattgccttatttctcactattgccttatttccgatcccggggtttaaaccgatcaacaaaatcactgatgctggacgatcaacaaaatcactgatgctggacgatcaacaaaatcactgatgctggatcaccaacaaaatcactgatgctggacgatgtttaaaccctgcaggcgatcagctatgccagcatcttgcccgatcagctatgccagcatcttgcccgatcagctatgccagcatcttgcctcaccagctatgccagcatcttgcccgatcctgcaggagatct

(19) United States
(12) Patent Application Publication
(10) Pub. No.: US 2026/0000790 A1
(43) Pub. Date: Jan. 1, 2026

(54) TRANSGENE CASSETTES

(71) Applicants: Ospedale San Raffaele S.r.l., Milan (IT); CONSIGLIO NAZIONALE DELLE RICERCHE, Rome (IT)

(72) Inventors: Vania Broccoli, Milan (IT); Mirko Luoni, Milan (IT)

(21) Appl. No.: 18/880,883

(22) PCT Filed: Jul. 7, 2023
(86) PCT No.: PCT/EP2023/068917

§ 371 Date: Jan. 3, 2025

(30) Foreign Application Priority Data
Jul. 8, 2022 (EP) 22183981.4

(51) Int. Cl. A61K 48/00; C12N 15/86

(52) U.S. Cl. CPC A61K 48/0058; A61K 48/0066; C12N 15/86; C12N 2750/14143

(57) ABSTRACT

A polynucleotide comprising at least one miR-124 target sequence, and/or at least one miR-338-3p target sequence, and/or at least one miR-31 target sequence, wherein the miRNA target sequences are operably linked to a transgene.

### FIELD OF THE INVENTION

The present invention relates to transgene cassettes and polynucleotides for the treatment of diseases or disorders. Polynucleotides of the invention may facilitate cell-specific transgene expression, for improved target specificity and safety.

### BACKGROUND TO THE INVENTION

Gene therapy involves the incorporation of genetic material into a cell to treat or prevent disease. The genetic material may supplement defective genes with functional copies of those genes, inactivate improperly functioning genes, silence genes that may be associated with a disease state or introduce new therapeutic genes to a cell.

A limitation in gene therapy is the ability to selectively determine whether a transgene is expressed within the cells to which it is delivered. There is an ongoing need for transgene expression cassettes that afford cell type specific transgene expression in order to reduce off-target effects associated with transgene expression, which in turn may improve the specificity and safety of any such gene therapies.

### SUMMARY OF THE INVENTION

Herein, the inventors provide a polynucleotide (e.g. transgene expression cassette) that utilizes miRNA target sequences in order to regulate transgene expression. Said expression cassettes allow transgene expression to be regulated such that unwanted expression is reduced or eliminated, thereby improving safety and reducing off target effects. The inventors have demonstrated the efficacy of their approach using a range of transgenes, such as GFP and epigenetic silencer factors (ESFs; engineered oncogenic and cancer-associated transcription factors that function as epigenetic repressors).

In a first aspect, there is provided a polynucleotide comprising at least one miR-124 target sequence, and/or at least one miR-338-3p target sequence, and/or at least one miR-31 target sequence, wherein the miRNA target sequences are operably linked to a transgene.

In one embodiment, the polynucleotide comprises at least one miR-124 target sequence, wherein the target sequence is operably linked to the transgene.

In one embodiment, the polynucleotide comprises at least one miR-338-3p target sequence, wherein the target sequence is operably linked to the transgene.

In one embodiment, the polynucleotide comprises at least one miR-31 target sequence, wherein the target sequence is operably linked to the transgene.

In one embodiment, the polynucleotide comprises at least one miR-124 target sequence, and at least one miR-338-3p target sequence, wherein the target sequences are operably linked to the transgene.

In one embodiment, the polynucleotide comprises at least one miR-124 target sequence, and at least one miR-31 target sequence, wherein the target sequences are operably linked to the transgene.

In one embodiment, the polynucleotide comprises at least one miR-338-3p target sequence, and at least one miR-31 target sequence, wherein the target sequences are operably linked to the transgene.

In one embodiment, the polynucleotide comprises at least one miR-124 target sequence, at least one miR-338-3p target sequence, and at least one miR-31 target sequence, wherein the target sequences are operably linked to the transgene.

In one aspect, there is provided a polynucleotide comprising at least one miR-124 target sequence, at least one miR-338-3p target sequence and at least one miR-31 target sequence, wherein the miRNA target sequences are operably linked to a transgene.

In one embodiment, the number of copies of each of the miRNA target sequences is independently selected from the group consisting of: one, two, three, and four.

In one embodiment, the polynucleotide comprises four miR-124 target sequences, four miR-338-3p target sequences and four miR-31 target sequences, wherein the miRNA target sequences are operably linked to the transgene.

In one embodiment:
(a) the miR-124 target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 1;
(b) the miR-338-3p target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 2; and/or
(c) the miR-31 target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 3.

In one embodiment, the miR-124 target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 1. In one embodiment, the miR-338-3p target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 2. In one embodiment, the miR-31 target sequence comprises or consists of a nucleotide sequence that has at least 90% sequence identity, such as at least 91%, 92%, 93%, 94%, 95%, 96%, 97%, 98%, 99%, or 100% sequence identity to SEQ ID NO: 3.

In one embodiment:
(a) the miR-124 target sequence comprises or consists of a nucleotide sequence that has at least 90% sequence identity to SEQ ID NO: 1;
(b) the miR-338-3p target sequence comprises or consists of a nucleotide sequence that has at least 90% sequence identity to SEQ ID NO: 2; and/or
(c) the miR-31 target sequence comprises or consists of a nucleotide sequence that has at least 90% sequence identity to SEQ ID NO: 3.

In one embodiment:
(a) the miR-124 target sequence comprises or consists of a nucleotide sequence that has at least 95% sequence identity to SEQ ID NO: 1;
(b) the miR-338-3p target sequence comprises or consists of a nucleotide sequence that has at least 95% sequence identity to SEQ ID NO: 2; and/or
(c) the miR-31 target sequence comprises or consists of a nucleotide sequence that has at least 95% sequence identity to SEQ ID NO: 3.

In one embodiment:
(a) the miR-124 target sequence comprises or consists of a nucleotide sequence that has at least 99% sequence identity to SEQ ID NO: 1;
(b) the miR-338-3p target sequence comprises or consists of a nucleotide sequence that has at least 99% sequence identity to SEQ ID NO: 2; and/or
(c) the miR-31 target sequence comprises or consists of a nucleotide sequence that has at least 99% sequence identity to SEQ ID NO: 3.

In one embodiment:
(a) the miR-124 target sequence comprises or consists of a nucleotide sequence that has 100% sequence identity to SEQ ID NO: 1;
(b) the miR-338-3p target sequence comprises or consists of a nucleotide sequence that has 100% sequence identity to SEQ ID NO: 2; and/or
(c) the miR-31 target sequence comprises or consists of a nucleotide sequence that has 100% sequence identity to SEQ ID NO: 3.

In one embodiment, the miRNA target sequences are located downstream, i.e., 3′, of the transgene. In other words, the miRNA target sequences may be located after the transgene in the 5′ to 3′ direction.

In one embodiment, the miRNA target sequences are located within the 3′-UTR of the transgene.

In one embodiment, the miRNA target sequences or clusters of copies of the miRNA target sequences are, from 5′ to 3′, arranged in the order: miR-124 target sequence(s), miR-338-3p target sequence(s), and miR-31 target sequence(s). The target sequences, or clusters comprising one or more copy thereof, may be, for example, arranged from 5′ to 3′ such that they form groups according to their target specificity, for example, in one embodiment the polynucleotide comprises 5′-[miR-124 target sequence]₄-[miR-338-3p target sequence]₄-[miR-31 target sequence]₄-3′.

Both the individual target sequences and the clusters of target sequences may be contiguous with one another, separated by spacer sequences, or any combination of thereof.

Thus, in one embodiment, the miRNA target sequences are separated by spacer sequences.

In one embodiment, there is provided a polynucleotide wherein the polynucleotide comprises a nucleotide sequence that has at least 90% sequence identity to SEQ ID NO: 4. In one embodiment, the polynucleotide comprises a nucleotide sequence that has at least 90%, such as at least 91%, 92%, 93%, 94%, 95%, 96%, 97%, 98%, 99%, or 100% sequence identity to SEQ ID NO: 4.

In one embodiment, the miRNA target sequences comprise a sequence as set forth in SEQ ID NO: 4.

In one embodiment, the miRNA target sequences consist of a sequence as set forth in SEQ ID NO: 4.

In one embodiment, the polynucleotide further comprises a promoter. In one embodiment, the promoter is operably linked to the transgene.

In one embodiment, the promoter is a constitutive promoter. In some embodiments, the constitutive promoter is an Ef1a promoter.

In one embodiment, the promoter is a tissue-specific promoter, preferably a cancer cell-specific promoter.

In one embodiment, the promoter is a proliferating cell-specific promoter.

In one embodiment, the polynucleotide further comprises a promoter operably linked to the transgene, optionally wherein the promoter is a tissue-specific promoter or a constitutive promoter, optionally a cancer cell-specific promoter.

In one embodiment, the promoter is selected from the group consisting of a Mki67 promoter, a Ccnd1 promoter, a Ccnb2 promoter, a Ccna2 promoter, a Cdc25c promoter, a Cdc2 promoter, a Cks1 promoter, a PCNA promoter, a CDC6 promoter, a POLD1 promoter, a CSK1B promoter, a MCM2 promoter and a PLK1 promoter.

In one embodiment, the promoter is a Mki67 promoter.

In one embodiment, the promoter is an Ef1a promoter.

In one aspect, there is provided a vector comprising the polynucleotide according to the invention.

In one embodiment, the vector is a viral vector.

In one embodiment, the vector is a lentiviral vector.

In one embodiment, the vector is an adeno-associated viral (AAV) vector.

In one embodiment, the AAV vector is of serotype, 2, 5, or 9. In a preferred embodiment, the vector is an AAV2 vector. In another preferred embodiment, the vector is an AAV5 vector.

In one embodiment, the vector is an mRNA vector.

The order of the miRNA target sequences may be varied. In one embodiment, the miRNA target sequences or clusters of copies of sequences are, from 5′ to 3′, arranged in the order: miR-124 target sequence(s), miR-338-3p target sequence(s), and miR-31 target sequence(s).

In one embodiment, the miRNA target sequences or clusters of copies of sequences are, from 5′ to 3′, arranged in the order: miR-124 target sequence(s), miR-31 target sequence(s), and miR-338-3p target sequence(s).

In another embodiment, the miRNA target sequences or clusters of copies of sequences are, from 5′ to 3′, arranged in the order: miR-338-3p target sequence(s), miR-124 target sequence(s), and miR-31 target sequence(s).

In another embodiment, the miRNA target sequences or clusters of copies of sequences are, from 5′ to 3′, arranged in the order: miR-338-3p target sequence(s), miR-31 target sequence(s), and miR-124 target sequence(s).

In another embodiment, the miRNA target sequences or clusters of copies of sequences are, from 5′ to 3′, arranged in the order: miR-31 target sequence(s), miR-124 target sequence(s), and miR-338-3p target sequence(s).

In another embodiment, the miRNA target sequences or clusters of copies of sequences are, from 5′ to 3′, arranged in the order: miR-31 target sequence(s), miR-338-3p target sequence(s), and miR-124 target sequence(s).

Both the individual target sequences and clusters of copies of sequences may be contiguous with one another, separated by spacer sequences, or any combination of thereof.

Thus, in one embodiment, the miRNA target sequences are separated by spacer sequences.

In one embodiment, there is provided a polynucleotide wherein the polynucleotide comprises a nucleotide sequence that has at least 90% sequence identity to SEQ ID NO: 4. In one embodiment, the polynucleotide comprises a sequence that has at least 90%, such as at least 91%, 92%, 93%, 94%, 95%, 96%, 97%, 98%, 99%, or 100% sequence identity to SEQ ID NO: 4.

In one aspect, there is provided the nanoparticle comprising a polynucleotide or vector according to the invention.

In one embodiment, the nanoparticle is a polymeric nanoparticle, inorganic nanoparticle or lipid nanoparticle. In some embodiments, the nanoparticle is a liposome

In one aspect, there is provided a cell comprising the polynucleotide, vector, or nanoparticle according to the invention.

In one embodiment, the cell is a eukaryotic cell, such as a mammalian cell. In another embodiment the cell is a human cell.

In one aspect, there is provided a composition comprising the polynucleotide, vector, nanoparticle, or cell according to the invention.

The composition may be a hydrogel. In some embodiments, the hydrogel is a poly(ethylene glycol) dimethacrylate (PEG-DMA) hydrogel. In some embodiments, the hydrogel further comprises hydroxyapatite nanoparticles.

In one aspect, there is provided the pharmaceutical composition comprising the polynucleotide, vector, nanoparticle, or cell according to the invention, and a pharmaceutically acceptable diluent, solvent, carrier, salt and/or adjuvant.

In one aspect, there is provided the polynucleotide, vector, nanoparticle, cell, composition, or pharmaceutical composition according to the invention, for use in therapy.

In one aspect, there is provided the polynucleotide, vector, nanoparticle, cell, composition, or pharmaceutical composition according to the invention, for use in the treatment of a disease or disorder.

In one aspect, there is provided the polynucleotide, vector, nanoparticle, cell, composition, or pharmaceutical composition according to the invention, for use in the treatment of cancer.

In some embodiments, the polynucleotide, vector, cell or composition (e.g. hydrogel) is administered locally.

In another aspect, the invention provides use of the polynucleotide, vector, nanoparticle, cell, composition, or pharmaceutical composition according to the invention, for the manufacture of a medicament for therapy.

In another aspect, the invention provides use of the polynucleotide, vector, nanoparticle, cell, composition, or pharmaceutical composition according to the invention, for the manufacture of a medicament for the treatment of cancer.

### DESCRIPTION OF THE DRAWINGS

FIG. 1. Enhancing transgene specificity using miRNA-based detargeting silencer factor constructs.

(a) Schematic depiction of a vector containing a Tmir cassette (four copies each of miRNA target sequences for miR124, miR338-3p, and miR31) within the 3′ UTR. The Tmir cassette allows the expression of the transgene in cancer cells but not in the brain cells (neurons, oligodendrocytes, and astrocytes). (b) GFP-Tmir is expressed in U251 GBM cancer cell line as indicated by GFP expression counterstained with Hoecst.

FIG. 2. AAV functionality on patient derived Cancer Stem Cells.

Recombinant AAV serotypes containing an Ef1a::GFP construct were used to infect patient derived GBM cancer stem cells in vitro. Four days after the infection, cells were fixed and immunofluorescence for GFP performed, and cells were stained with the nuclear marker Hoecst.

FIG. 3. Enhancing ESF specificity using miRNA-based detargeting silencer factor constructs.

(a) Schematic depiction of both the control and SES vector containing a Tmir cassette (four copies each of miRNA target sequences for miR124, miR338-3p, and miR31) within the 3′ UTR. The Tmir cassette allows the expression of the transgene in cancer cells but not in the brain cells (neurons, oligodendrocytes, and astrocytes). (b) Both GFP-Tmir and SES-Tmir are expressed in U251 GBM cancer cell line as indicated by GFP/V5 expression counterstained with Hoecst. SES-Tmir is able to reduce the proliferation of cancer cell in vitro. (c) SES-Tmir is not expressed (detargeted) in mouse primary cortical cultures that contain post-mitotic neurons, post-mitotic and proliferating astrocytes as well as proliferating Oligo Precursor Cells (OPCs).

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

It will be understood that when referring to a protein or polypeptide herein, the same may equally be applied to a polynucleotide encoding the same, and, where relevant (i.e., when referring to a coding sequence within a polynucleotide) vice versa.

It will be understood that any of the following aspects of the invention may be suitably combined in the practice of the invention herein.

### Polynucleotides and Transgene Cassettes

Triplet miRNA Cassettes

Herein, the inventors provide a transgene expression cassette that comprises target sequences that are recognized by micro RNAs (miRNAs), in order to regulate transgene expression. Said expression cassettes allow the expression of a transgene to be regulated such that unwanted expression is reduced or eliminated in cell types comprising the miRNA, thereby improving safety and reducing off target effects.

In one aspect, there is provided a polynucleotide comprising at least one miR-124 target sequence, at least one miR-338-3p target sequence and at least one miR-31 target sequence, wherein the miRNA target sequences are operably linked to a transgene.

In one embodiment, the number of copies of each of the miRNA target sequences is independently selected from the group consisting of: one, two, three, and four.

In one embodiment, the number of copies of each of the miRNA target sequences is one.

In one embodiment, the number of copies of each of the miRNA target sequences is two.

In one embodiment, the number of copies of each of the miRNA target sequences is three.

In one embodiment, the number of copies of each of the miRNA target sequences is four.

In one embodiment, the number of copies of each of the miRNA target sequences is greater than four, such as five, six, seven, eight, nine, or ten.

A suitable miRNA target sequence for miR-124 is:
attgccttatttc [SEQ ID NO: 1]

In one embodiment, the miRNA target sequence comprises the sequence of SEQ ID NO: 1.

A suitable miRNA target sequence for miR-338-3p is:
caacaaaatcactgatgctgga [SEQ ID NO: 2]

In one embodiment, the miRNA target sequence comprises the sequence of SEQ ID NO: 2.

A suitable miRNA target sequence for miR-31 is:

[SEQ ID NO: 3]
cagctatgccagcatcttgcc

In one embodiment, the miRNA target sequence comprises the sequence of SEQ ID NO: 3.

In one embodiment, the miRNA target sequences comprise SEQ ID NOs: 1, 2, and 3.

In one embodiment, the miRNA target sequences are located downstream, i.e., 3′, of the transgene.

In one embodiment, the miRNA target sequences are located within the 3′-UTR of the transgene.

In one embodiment, the miRNA target sequences or clusters of copies of sequences are, from 5′ to 3′, arranged in the order: miR-124, miR-338-3p, and miR-31.

According to the foregoing embodiment, the target sequences, or clusters comprising one or more copy thereof, are arranged from 5′ to 3′ such that they form groups according to their target specificity, i.e., 5′-[miR-124 target sequence]₄-[miR-338-3p target sequence]₄-[miR-31 target sequence]₄-3′.

Both the individual target sequences and the clusters of sequences may be contiguous with one another, separated by spacer sequences, or any combination thereof.

Thus, in one embodiment, the miRNA target sequences are separated by spacer sequences.

In an embodiment wherein the polynucleotide comprises four target sequences for each of miR-124, miR-338-3p, and miR-31, the polynucleotide may comprise the sequence as set forth in SEQ ID NO: 4.

Triple miRNA Target sequence (Tmir) (SEQ ID NO: 4):
atattgccttatttccgattattgccttatttcgcattattgccttatttctcactattgccttattt

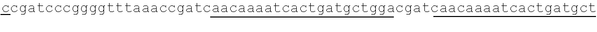

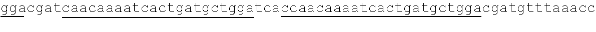

ctgcaggcgatcagctatgccagcatcttgcccgatcagctatgccagcatcttgcccgatcagctat
gccagcatcttgcctcaccagctatgccagcatcttgcccgatcctgcaggagatct
miR124 Target sequences

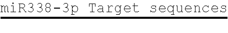

miR31 Target sequences

In one embodiment, the polynucleotide comprises a sequence as set forth in SEQ ID NO: 4.

In one embodiment, the polynucleotide consists of a sequence as set forth in SEQ ID NO: 4.

The polynucleotide and transgene cassette described herein may be used with any transgene.

miR-124 may be, for example, also referred to as miRNA-124 or miR124; miR-338-3p, as miR-338-3p, or miRNA338-3p; and miR-31 as miRNA-31 or miR31.

miRNA target sequences may be represented by one of more copies of a sequence of a given identity. The number of copies of each target sequence may be independently selected, i.e., the number of copies of a given sequence is not necessarily dependent on the number of copies of another, different, sequence.

Thus, in one embodiment, the miRNA target sequence(s) comprises more than one copy of said miRNA target sequence.

In one embodiment, the miRNA target sequence(s) comprises one copy of said miRNA target sequence.

In one embodiment, the miRNA target sequence(s) comprises two copies of said miRNA target sequence.

In one embodiment, the miRNA target sequence(s) comprises three copies of said miRNA target sequence.

In one embodiment, the miRNA target sequence(s) comprises four copies of said miRNA target sequence.

In one embodiment, the miRNA target sequence(s) comprises more than four copies, for example five, six, seven, eight, nine, or ten copies of said miRNA target sequence.

In a preferred embodiment, the miRNA target sequences comprise four copies of a target sequence for each of miR-124, miR-338-3p, and miR-31.

The order of the miRNA target sequences may be varied. In one embodiment, the miRNA target sequences or clusters of copies of sequences are, from 5′ to 3′, arranged in the order: miR-124, miR-338-3p, and miR-31.

In another embodiment, the miRNA target sequences or clusters of copies of sequences are, from 5′ to 3′, arranged in the order: miR-124, miR-31, and miR-338-3p.

In another embodiment, the miRNA target sequences or clusters of copies of sequences are, from 5′ to 3′, arranged in the order: miR-338-3p, miR-124, and miR-31.

In another embodiment, the miRNA target sequences or clusters of copies of sequences are, from 5′ to 3′, arranged in the order: miR-338-3p, miR-31, and miR-124.

In another embodiment, the miRNA target sequences or clusters of copies of sequences are, from 5′ to 3′, arranged in the order: miR-31, miR-124, and miR-338-3p.

In another embodiment, the miRNA target sequences or clusters of copies of sequences are, from 5′ to 3′, arranged in the order: miR-31, miR-338-3p, and miR-124.

Both the individual target sequences and the clusters of sequences may be contiguous with one another, separated by spacer sequences, or any combination thereof.

Thus, in one embodiment, the miRNA target sequences are separated by spacer sequences.

In one embodiment, the polynucleotide comprises a sequence according to SEQ ID NO: 4.

### Spacers

As used herein, a “spacer” may be a sequence (e.g. a nucleotide or amino acid sequence) that may be used to separate other sequence elements within a larger polymer.

In one embodiment, one or more spacer sequence separates polynucleotide sequences (e.g. miRNA target sequences).

Individual miRNA target sequences or groups of miRNA target sequences may be separated by one or more spacer sequence. In one embodiment, the miRNA target sequences are separated by one or more spacer sequence. The spacer sequence may comprise, for example, at least one, at least two, at least three, at least four, at least five, at least ten, at least twenty, or at least thirty nucleotide bases.

By way of non-limiting example, in the following Triple miRNA Target sequence, the miRNA target sequences are each separated by sequences which may not be considered part of said miRNA target sequence. Such sequences may be considered to be spacers that separate functional sequence elements.

Triple miRNA Target sequence (Tmir) (SEQ ID NO: 4):
atattgccttatttccgattattgccttatttcgcattattgccttatttctcactattgccttattt

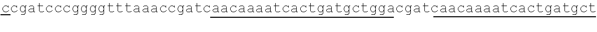

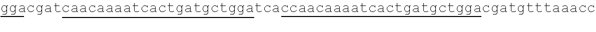

ctgcaggcgatcagctatgccagcatcttgcccgatcagctatgccagcatcttgcccgatcagctat
gccagcatcttgcctcaccagctatgccagcatcttgcccgatcctgcaggagatct

In one embodiment, the spacer is selected from the group consisting of: (a)-(i).

In one embodiment, the polynucleotide comprises one or more spacer selected from the group consisting of: (a)-(i).

In one embodiment, the spacer sequences separating the clusters of miRNA target sequences are longer than the spacer sequences separating the miRNA target sequences within a cluster.

### Transgenes

The polynucleotide according to the invention may comprise any suitable transgene. A suitable transgene may be operably linked to the miRNA target sequences according to the invention such that the expression of said transgene is dependent upon the presence of the miRNA.

By “operably linked”, it is to be understood that individual components are linked together in a manner which enables them to carry out their function substantially unhindered

### Expression Control Sequences

The polynucleotide of the invention may comprise one or more expression control sequence. Suitably, the transgene is operably linked to one or more expression control sequence.

As used herein an “expression control sequence” is any nucleotide sequence which controls expression of a transgene, e.g. to facilitate and/or increase expression in some cell types and/or decrease expression in other cell types.

The expression control sequence and the transgene may be in any suitable arrangement in the polynucleotide, providing that the expression control sequence is operably linked to the transgene.

### Promoters

In some embodiments, the expression control sequence is a promoter.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, the polynucleotide further comprises a promoter operably linked to the transgene.

In some embodiments, the promoter is a constitutive promoter. In some embodiments, the constitutive promoter is an Ef1a promoter.

An example sequence of an Ef1a promoter is:

(SEQ ID NO: 8)
CGATAAGCTTTGCAAAGATGGATAAAGTTTTAAACAGAGAGGAATCTTTGCAGCTAATGGAC
CTTCTAGGTCTTGAAAGGAGTGGGAATTGGCTCCGGTGCCCGTCAGTGGGCAGAGCGCACAT
CGCCCACAGTCCCCGAGAAGTTGGGGGGAGGGGTCGGCAATTGAACCGGTGCCTAGAGAAGG
TGGCGCGGGGTAAACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCAACGGGTTTGCCG
CCAGAACACAGGTAAGTGCCGTGTGTGGTTCCCGCGGGCCTGGCCTCTTTACGGGTTATGGC
CCTTGCGTGCCTTGAATTACTTCCACTGGCTGCAGTACGTGATTCTTGATCCCGAGCTTCGG
GTTGGAAGTGGGTGGGAGAGTTCGAGGCCTTGCGCTTAAGGAGCCCCTTCGCCTCGTGCTTG
AGTTGAGGCCTGGCCTGGGCGCTGGGGCCGCCGCGTGCGAATCTGGTGGCACCTTCGCGCCT
GTCTCGCTGCTTTCGATAAGTCTCTAGCCATTTAAAATTTTTGATGACCTGCTGCGACGCTT
TTTTTCTGGCAAGATAGTCTTGTAAATGCGGGCCAAGATCTGCACACTGGTATTTCGGTTTT
TGGGGCCGCGGGCGGCGACGGGGCCCGTGCGTCCCAGCGCACATGTTCGGCGAGGCGGGGCC
TGCGAGCGCGGCCACCGAGAATCGGACGGGGGTAGTCTCAAGCTGGCCGGCCTGCTCTGGTG
CCTGGCCTCGCGCCGCCGTGTATCGCCCCGCCCTGGGCGGCAAGGCTGGCCCGGTCGGCACC
AGTTGCGTGAGCGGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGGGCCTTTCCGTCC
TCAGCCGTCGCTTCATGTGACTCCACGGAGTACCGGGCGCCGTCCAGGCACCTCGATTAGTT
CTCGAGCTTTTGGAGTACGTCGTCTTTAGGTTGGGGGGAGGGGTTTTATGCGATGGAGTTTC
CCCACACTGAGTGGGTGGAGACTGAAGTTAGGCCAGCTTGGCACTTGATGTAATTCTCCTTG
GAATTTGCCCTTTTTGAGTTTGGATCTTGGTTCATTCTCAAGCCTCAGACAGTGGTTCAAAG
TTTTTTTCTTCCATTTCAGGTGTCGTGAGGAATTCTGCAGTCGATCGACGGTACCGCGGGCC
C

In some embodiments, the promoter is a tissue-specific promoter, preferably a cancer cell-specific promoter.

In some embodiments, the promoter is a proliferating cell-specific promoter.

In some embodiments, the promoter is selected from the group consisting of a Mki67 promoter, a Ccnd1 promoter, a Ccnb2 promoter, a Ccna2 promoter, a Cdc25c promoter, a Cdc2 promoter, a Cks1 promoter, a PCNA promoter, a CDC6 promoter, a POLD1 promoter, a CSK1B promoter, a MCM2 promoter and a PLK1 promoter.

In some embodiments, the promoter is a Mki67 promoter.

An example sequence of a Mki67 promoter is:

(SEQ ID NO: 9)
acttcaggcttgtccttgagaccttgaggagagggtagacattggttctattgccccagaag
cggaatttttacaacaccctactggaagaacactgatccaggaacaggaggagggagcatga
aaaaagaggacaggaaaacataggtactctatattcatctgactggcacctggcctcagata
accccaactggtgccccaggagccctattttaggatttgcaggtgcagtcttgcacaggtta
ttttaatcggcatttccccctgaaacagcaaagcattgcaaacttaaccgtgccgcagcaat
aactcttaagttatggtttcggaaaaaccgtgccactgcctcagtgttcattaagcagggag
actgtagaatgaattataatgatgtcccccagtttttaaaggacagtttgagggatactgaa
aaggcaggagaaacaaccttaggttgcgtatgaggtgttgcagaaacggcagaggaagggta
taaattttaaaaatagaaagcttctagcgttgagcaagatgcggcgttgtcggtgtttgatt
tctggagagggagcctacaggtagcttcctacaggtcgccacgctagttttgctgacactga
aaaagctgggaccctctaccctgcctattagagcagcagtggcccgcccaccagccccaccc
tgcccgccttccggagcttatctccgctcgggttccgcttctccagccagcttctggcccgg
ccccgcctaccggcctggcccctcccaccttccggctccacccccccccccccccccaggct
cgcagcctggccccacctctggacttccccaatcctctgtcgccctcgccattggtctctgg
ctggccgcctggcccaatcgcagcacttagcgccagaatttgaaccgccgttttggtttgaa
tcgggcgggctgcggcgggcgagccttggcgcggaaccgcctaggcggacagtcggggctgg
acggggcggcgggtggcgggtggcgggcacttgggacgcgggcggcgggagtgcgggtctgg
tcggggcggagcgaaggccgcgggtggccgtggtcggtcctccgcggctaaggagccgaggg
ctccgacgcgggctgcgcccggtgagcggcggccagagctaacttgcgctgactggaccagc
tgaggagcggcccggcggggcgactgcgagcttcaccgagaggcttctccgccctggtccgc
agtcccgacggccgggcggacc

The promoter may, for example, comprise or consist of a nucleic acid sequence that has at least 70%, 80%, 90%, 95%, 96%, 97%, 98%, 99% or 100% sequence identity to any one of SEQ ID NOs: 8 or 9, preferably wherein the promoter substantially retains the natural function of the promoter of SEQ ID NO: 8 or 9, respectively.

miRNA Target Sequences

In some embodiments, the polynucleotide further comprises one or more miRNA target sequence. Suitably, the nucleic acid sequence encoding the transgene is operably linked to the one or more miRNA target sequence.

MicroRNA (miRNA) genes are scattered across all human chromosomes, except for the Y chromosome. They can be either located in non-coding regions of the genome or within introns of protein-coding genes. Around 50% of miRNAs appear in clusters which are transcribed as polycistronic primary transcripts. Similar to protein-coding genes, miRNAs are usually transcribed from polymerase-Il promoters, generating a so-called primary miRNA transcript (pri-miRNA). This pri-miRNA is then processed through a series of endonucleolytic cleavage steps, performed by two enzymes belonging to the RNAse Type Ill family, Drosha and Dicer. From the pri-miRNA, a stem loop of about 60 nucleotides in length, called miRNA precursor (pre-miRNA), is excised by a specific nuclear complex, composed of Drosha and DiGeorge syndrome critical region gene (DGCR8), which crops both strands near the base of the primary stem loop and leaves a 5′ phosphate and a 2 bp long, 3′ overhang. The pre-miRNA is then actively transported from the nucleus to the cytoplasm by RAN-GTP and Exportin. Then, Dicer performs a double strand cut at the end of the stem loop not defined by the Drosha cut, generating a 19-24 bp duplex, which is composed of the mature miRNA and the opposite strand of the duplex, called miRNA*. In agreement with the thermodynamic asymmetry rule, only one strand of the duplex is selectively loaded into the RNA-induced silencing complex (RISC), and accumulates as the mature microRNA. This strand is usually the one whose 5′ end is less tightly paired to its complement, as was demonstrated by single-nucleotide mismatches introduced into the 5′ end of each strand of siRNA duplexes. However, there are some miRNAs that support accumulation of both duplex strands to similar extent.

MicroRNAs trigger RNAi, very much like small interfering RNAs (siRNA) which are extensively used for experimental gene knockdown. The main difference between miRNA and siRNA is their biogenesis. Once loaded into RISC, the guide strand of the small RNA molecule interacts with mRNA target sequences preferentially found in the 3′ untranslated region (3′UTR) of protein-coding genes. It has been shown that nucleotides 2-8 counted from the 5′ end of the miRNA, the so-called seed sequence, are essential for triggering RNAi. If the whole guide strand sequence is perfectly complementary to the mRNA target, as is usually the case for siRNAs and plant miRNAs, the mRNA is endonucleolytically cleaved by involvement of the Argonaute (Ago) protein, also called “slicer” of the small RNA duplex into the RNA-induced silencing complex (RISC). DGRC (DiGeorge syndrome critical region gene 8) and TRBP (TAR (HIV) RNA binding protein 2) are double-stranded RNA-binding proteins that facilitate mature miRNA biogenesis by Drosha and Dicer RNase Ill enzymes, respectively. The guide strand of the miRNA duplex gets incorporated into the effector complex RISC, which recognises specific targets through imperfect base-pairing and induces post-transcriptional gene silencing. Several mechanisms have been proposed for this mode of regulation: miRNAs can induce the repression of translation initiation, mark target mRNAs for degradation by deadenylation, or sequester targets into the cytoplasmic P-body.

On the other hand, if only the seed is perfectly complementary to the target mRNA but the remaining bases show incomplete pairing, RNAi acts through multiple mechanisms leading to translational repression. Eukaryotic mRNA degradation mainly occurs through the shortening of the polyA tail at the 3′ end of the mRNA, and de-capping at the 5′ end, followed by 5′-3′ exonuclease digestion and accumulation of the miRNA in discrete cytoplasmic areas, the so called P-bodies, enriched in components of the mRNA decay pathway.

Expression of the nucleic acid sequence encoding the transgene may be regulated by one or more endogenous miRNAs using one or more corresponding miRNA target sequence. Using this method, one or more miRNAs endogenously expressed in a cell prevent or reduce transgene expression in that cell by binding to its corresponding miRNA target sequence positioned in the polynucleotide or vector.

The target sequence may be fully or partially complementary to the miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA. Suitably, a partially complementary miRNA target sequence may be fully complementary to the miRNA seed sequence.

Including more than one copy of a miRNA target sequence may increase the effectiveness of the system. Also, different miRNA target sequences can be included. For example, the protein-coding sequence may be operably linked to more than one miRNA target sequence, which may or may not be different. The miRNA target sequences may be in tandem, but other arrangements are envisaged. The polynucleotide may, for example, comprise 1, 2, 3, 4, 5, 6, 7 or 8 copies of the same or different miRNA target sequences. Suitably, the polynucleotide comprises 4 copies of each miRNA target sequence.

Copies of miRNA target sequences may be separated by a spacer sequence. The spacer sequence may comprise, for example, at least one, at least two, at least three, at least four or at least five nucleotide bases.

The one or more miRNA target sequence may, for example, suppress expression of the transgene in non-cancer cells. This may, for example, increase safety of a therapy targeting the cancer cells. Expression of the transgene in cancer cells may, for example, not be suppressed by the one or more miRNA target sequence.

The one or more miRNA target sequence may suppress transgene expression in one or more cells other than cancer cells, for example neurons, astrocytes and/or oligodendrocytes. In one embodiment, the one or more miRNA target sequence suppresses transgene expression in neurons. In one embodiment, the one or more miRNA target sequence suppresses transgene expression in astrocytes. In one embodiment, the one or more miRNA target sequence suppresses transgene expression in oligodendrocytes.

The term “suppress expression” as used herein may refer to a reduction of expression in the relevant cell type(s) of a transgene to which the one or more miRNA target sequence is operably linked as compared to transgene expression in the absence of the one or more miRNA target sequence, but under otherwise substantially identical conditions. In some embodiments, transgene expression is suppressed by at least 50%. In some embodiments, transgene expression is suppressed by at least 60%, 70%, 80%, 90% or 95%. In some embodiments, transgene expression is substantially prevented.

### Proteins

The term “protein” as used herein includes single-chain polypeptide molecules as well as multiple-polypeptide complexes where individual constituent polypeptides are linked by covalent or non-covalent means. The terms “polypeptide” and “peptide” as used herein refer to a polymer in which the monomers are amino acids and are joined together through peptide or disulfide bonds.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

Transgenes and coding sequences of the invention, such as sequences disclosed herein, may also include a stop codon, for example TGA, at the 3′ end of the transgene or coding sequence.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to the skilled person. They may also be cloned by standard techniques.

Longer polynucleotides will generally be produced using recombinant means, for example using polymerase chain reaction (PCR) cloning techniques. This may involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides used in the invention may be introduced into cells using a variety of techniques known in the art, such as transfection, transformation and transduction. Several such techniques are known in the art, for example infection with recombinant viral vectors, such as retroviral, lentiviral (e.g. integration-defective lentiviral), adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Transfection of cells with mRNA vectors can be achieved, for example, using nanoparticles, such as liposomes.

In some embodiments, the vector (e.g. mRNA vector) is comprised in a nanoparticle. In some embodiments, the nanoparticle is a polymeric nanoparticle, inorganic nanoparticle or lipid nanoparticle. In some embodiments, the nanoparticle is a liposome.

The nanoparticle may be targeted to a specific cell type(s) (e.g. cancer cells) using one or more ligand displayed on its surface.

### Viral Vectors

In preferred embodiments, the vector is a viral vector. The viral vector may be in the form of a viral vector particle.

The viral vector may be, for example, a retroviral, lentiviral, adeno-associated viral (AAV) or adenoviral vector.

In some embodiments, the vector is a lentiviral vector. In some embodiments, the vector is an AAV vector. In some embodiments, the vector is an AAV vector particle.

### Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective.

Lentivirus vectors are part of the larger group of retroviral vectors. A detailed list of lentiviruses may be found in Coffin et al. (1997) Retroviruses, Cold Spring Harbour Laboratory Press, 758-63. Lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis et al. (1992) EMBO J. 11: 3053-8; Lewis et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site) or by modifying or deleting essential att sequences from the vector LTR, or by a combination of the above.

### Adeno-Associated Viral (AAV) Vectors

Adeno-associated virus (AAV) is an attractive vector system for use in the invention as it has a high frequency of integration. Furthermore, AAVs can spread through the brain tissue due to their small size and reduced binding to cell membranes.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

In some embodiments the vector is an AAV2 vector. In some embodiments the vector is an AAV5 vector.

In some embodiments the vector is an AAV9 vector.

The viral vectors may be modified or mutant viral vectors. Such vectors may be vectors modified to possess certain desirable properties. For example, the AAV2 vector HBKO (or AAV2-HBKO) is a modified AAV2 that is incapable of binding to the heparin sulfate proteoglycan receptor (Naidoo et al. (2018) Mol Ther 26: 2418-2430).

In one embodiment, the vector is a modified AAV2 vector.

In one embodiment, the vector is an AAV2-HBKO vector.

Modified viral vectors may comprise proteins that confer, for example, altered cell tropism, and have been described, for example, in WO2021155137 and WO2015168666.

In one embodiment, the vector is a modified AAV5 vector.

In one embodiment, the vector comprises a capsid with one or more mutations in one or more capsid proteins.

In one embodiment, the vector comprises a capsid with one or more mutations in VP1.

In one embodiment, the vector is an AAV vector, preferably an AAV5 vector, that comprises a capsid protein (e.g. a VP1 protein) comprising: (a) a G at the position corresponding to amino acid 194; (b) an R at the position corresponding to amino acid 474; (c) an R at the position corresponding to amino acid 564; and/or (d) an R at the position corresponding to amino acid 573, wherein the amino acids are numbered with reference to VP1 of AAV5.

### Variants, Derivatives, Analogues, Homologues, and Fragments

In addition to the specific proteins and polynucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

The term “derivative” as used herein in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

A homologous sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs disclosed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package, FASTA (Atschul et al. (1990) J. Mol. Biol. 403-410) and the GENEWORKS suite of comparison tools. Both BLAST and FASTA are available for offline and online searching. However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### Compositions

The polynucleotides, vectors, nanoparticles and cells of the invention may be formulated for administration to subjects with a pharmaceutically-acceptable carrier, diluent or excipient. Suitable carriers and diluents include isotonic saline solutions, for example phosphate-buffered saline, and potentially contain human serum albumin.

Materials used to formulate a pharmaceutical composition should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

### Methods of Treatment

In one aspect, the invention provides the polynucleotide, vector, nanoparticle, cell, composition or pharmaceutical composition of the invention for use in therapy.

In another aspect, the invention provides the polynucleotide, vector, cell or composition of the invention for use in the treatment of cancer.

All references herein to treatment include curative, palliative and prophylactic treatment. The treatment of mammals, particularly humans, is preferred. Both human and veterinary treatments are within the scope of the invention.

In some embodiments, the method of treatment provides the polynucleotide, vector, nanoparticle, cell, composition or pharmaceutical composition of the invention to a tumor.

In some embodiments, the method of treatment provides the polynucleotide, vector, nanoparticle, cell, composition or pharmaceutical composition of the invention to the brain of a subject.

### Administration

In some embodiments, the polynucleotide, vector, nanoparticle, cell, composition or pharmaceutical composition is administered to a subject locally.

In some embodiments, the polynucleotide, vector, nanoparticle, cell, composition or pharmaceutical composition is administered to a subject's brain.

In preferred embodiments, the polynucleotide, vector, nanoparticle, cell, composition or pharmaceutical composition is administered to a tumor.

In some embodiments, the polynucleotide, vector, nanoparticle, cell, composition or pharmaceutical composition is administered to a subject systemically, for example intravenously.

In some embodiments, the polynucleotide, vector, nanoparticle, cell, composition or pharmaceutical composition is administered to a subject locally.

The term “systemic delivery” or “systemic administration” as used herein means that the agent of the invention is administered into the circulatory system, for example to achieve broad distribution of the agent. In contrast, topical or local administration restricts the delivery of the agent to a localised area, e.g. a tumor.

### Dosage

The skilled person can readily determine an appropriate dose of an agent of the invention to administer to a subject. Typically, a physician will determine the actual dosage that will be most suitable for an individual patient, which will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

All publications mentioned in the above specification are herein incorporated by reference. Various modifications and variations of the disclosed polypeptides, polynucleotides, vectors, cells, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### EXAMPLES

### Example 1

### Material and Methods

atattgccttatttccgattattgccttatttcgcattattgccttatttctcactattgccttattt

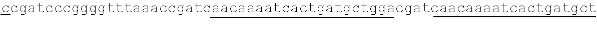

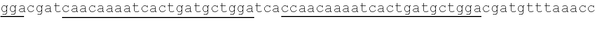

ctgcaggcgatcagctatgccagcatcttgcccgatcagctatgccagcatcttgcccgatcagctat
gccagcatcttgcctcaccagctatgccagcatcttgcccgatcctgcaggagatct
miR124 Target sequences

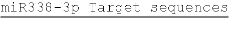

miR31 Target sequences

### Cell Culture

U-251 cells were cultured in plastic-adherence conditions in DMEM medium (Dulbecco's Modified Eagle's Medium—high glucose, Sigma-Aldrich) containing 10% fetal bovine serum (FBS, Sigma-Aldrich), 1% Pen/Strept (Sigma-Aldrich), 2 mM Glutamine (Sigma-Aldrich), 1% non-essential amino acids (MEM NEAA, ThermoFisher Scientific), 1% sodium pyruvate solution (Sigma-Aldrich) and passaged twice a week using Trypsin-EDTA solution (Sigma-Aldrich).

Cancer stem cells (CSCs) glioblastoma tumors were maintained in plastic-adherence conditions in DMEM/F12 (Sigma-Aldrich) supplemented with Hormon Mix (DMEM/F12, 0.6% Glucose (Sigma-Aldrich) (30% in phosphate buffer (PBS) (Euroclone)), Insulin (Sigma-Aldrich) 250 μg/ml, putrescine powder (Sigma-Aldrich) 97 μg/ml, apotransferrin powder (Sigma Aldrich), sodium selenite 0.3 μM, progesterone 0.2 μM), 1% Pen/Strept, 2 mM Glutamine, 0.66% Glucose (30% in phosphate buffer salt (PBS) (Euroclone)), and heparin (4 mg/ml, Sigma-Aldrich); bFGF (20 ng/ml, ThermoFisher Scientific) and EGF (20 ng/ml, ThermoFisher Scientific) were added freshly to culture medium.

All the cultures were kept in humidified atmosphere of 5% CO2 at 37° C. under atmospheric oxygen conditions.

### Immunostaining

Cells were seeded on glass coverslips and they were fixed for 20 minutes on ice in 4% paraformaldehyde (PFA, Sigma), solution in phosphate-buffered saline (PBS, Euroclone). Then they were washed twice with PBS and were permeabilized for 30′ in blocking solution, containing 0.2% Triton X-100 (SigmaAldrich) and 5% donkey serum (Euroclone), and incubated overnight at 4° C. with the primary antibodies diluted in blocking solution. The primary antibodies utilized were as follows: anti-V5 (mouse, 1:500, ThermoFisher Scientific, R96025), anti-GFP (chicken, 1:1000, Thermo Fisher Scientific, A10262), and anti-MAP2 (chicken, 1:1000, Abcam, ab92434). The next day, cells were washed 3 times with PBS for 5 minutes and incubated for 1 hour at room temperature with Hoechst 33342 (ThermoFischer Scientific) and with secondary antibodies (ThermoFisher Scientific) in blocking solution. Finally, slides were washed and mounted in Fluorescent Mounting Medium (Dako Cytomation). Images were acquired with epifluorescence microscope Nikon DS-Qi2 and analyzed with Fiji software.

### AAV Production and Infection

Replication-incompetent, recombinant viral particles were produced in 293 T cells by polyethylenimine (PEI) (Polyscience) co-transfection of three different plasmids: transgene-containing plasmid, packaging plasmid for rep and cap genes and pHelper (Agilent) for the three adenoviral helper genes. The cells and supernatant were harvested at 120 hr. Cells were lysed in hypertonic buffer (40 mM Tris, 500 mM NaCl, 2 mM MgCl2, pH=8) containing 100 U/ml Salt Active Nuclease (SAN, Arcticzymes) for 1 hr at 37° C., whereas the viral particles present in the supernatant were concentrated by precipitation with 8% PEG8000 (Polyethylene glycol 8000, Sigma-Aldrich) and then added to supernatant for an additional incubation of 30 min at 37° C. To clarify the lysate cellular debris were separated by centrifugation (4000 g, 30 min). The viral phase was isolated by iodixanol step gradient (15%, 25%, 40%, 60% Optiprep, Sigma-Aldrich) in the 40% fraction and concentrated in PBS (Phosphate Buffer Saline) with 100K cut-off concentrator (Amicon Ultra15, MERCK-Millipore). Virus titers were determined using AAVpro Titration Kit Ver2 (TaKaRa). 2.5×10∝CSCs L0627 were infected with adenoviral vectors expressing GFP (5 μl/well) and seeded in a 24 multi-wells plate on glass coverslips previously coated with Matrigel. After 4 days, cells were fixed and used for immunostaining studies.

### Results

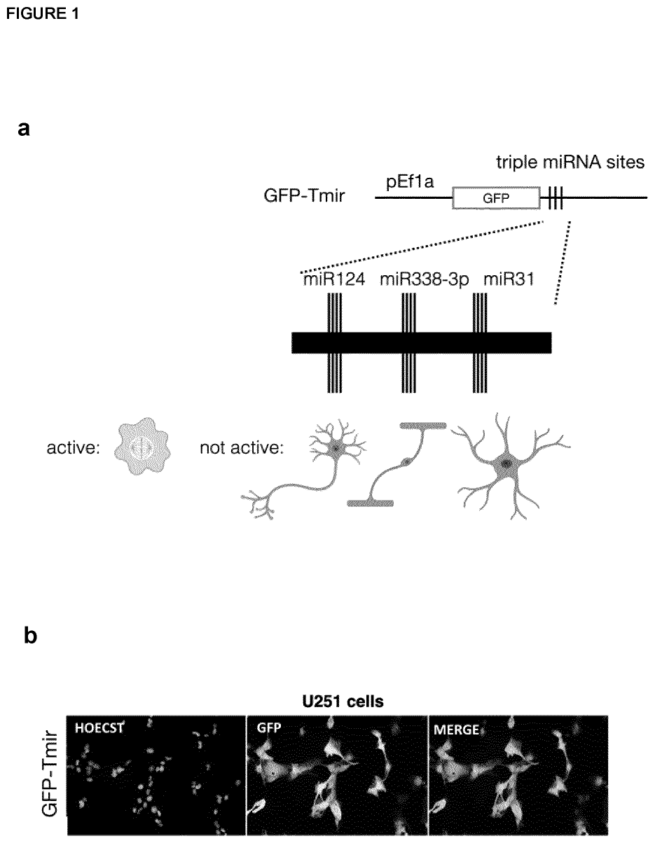

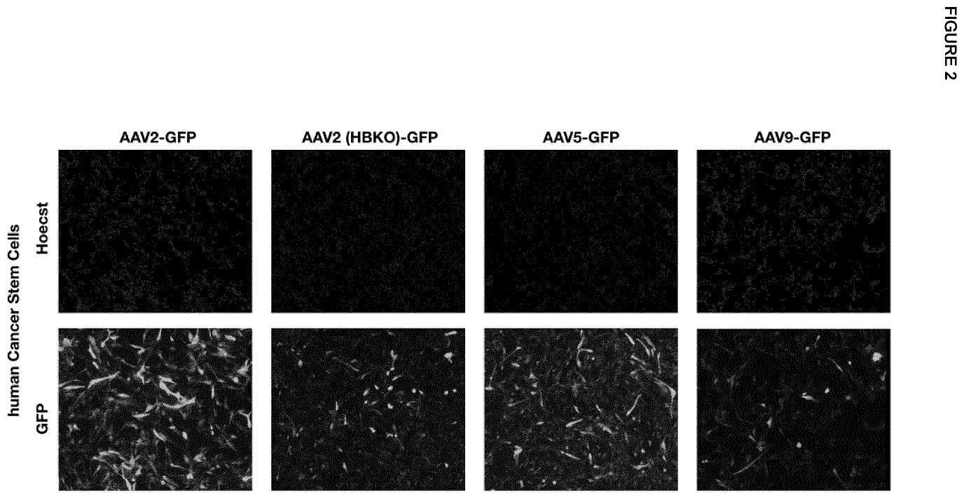

The inventors have devised a strategy to restrict transgene expression to certain cells, for example after viral inoculation of the brain. Said strategy is based on a microRNA (miRNAs) detargeting system, which enables the silencing of a transgene by the inclusion of binding/target sites (TS) of miRNAs endogenously expressed in specific cell types in which expression is not desired. To this end, a cassette was generated in which the 3′-UTR downstream to the transgene, comprised a cluster of 4×TS for each of miRNA-124, -338-3p and -31, which are specifically expressed in neurons, astrocytes and oligodendrocytes, respectively. Thus, exogenous transgene expression, e.g., GFP expression as demonstrated in FIG. 1, should be silenced in all of the foregoing cell types through miRNA-dependent post-transcriptional silencing and block of protein translation (FIG. 1a). Notably, miRNA-124, -338-3p and -31, are not expressed in GBM, as demonstrated by GFP-Tmir lentiviruses (LVs), which exhibited unaffected transgene expression in cancer cells (FIG. 1b).

It may be desirable that transgenes be used with a system of delivery that allows easy and broad distribution of the vector over the brain area that may contain residual tumor cells after surgery. This would reduce the likelihood of tumor recurrence, which in GBM is a significant problem. To this end, the inventors tested the use of adeno-associated viruses (AAVs) as shuttling vectors. Recombinant serotypes 2 and 5 performed better, as compared to serotype 9, for transgene delivery, as assessed by GFP expression, within patient derived cancer stem cells (FIG. 2).

### Discussion

The inventors herein demonstrate detargeting the expression of exogenous transgenes from healthy brain cells, e.g., neurons, astrocytes and oligodendrocytes, by using miRNA target sequences within the 3′ UTR, thus affording improved safety and specificity. By utilizing target sequences of miRNAs that are highly expressed in brain cells, but not in tumor cells, the inventors provide polynucleotides and transgene expression cassettes with high specificity, e.g., for use as an anti-GBM treatment.

The inventors herein also provide alternative therapeutic viruses to lentiviruses, that could be similarly used. Particularly useful are strains of adeno-associated viruses (AAVs) that can spread through the brain tissue due to their small size and which exhibit reduced binding to cell membranes. Maximizing viral spreading in the brain parenchyma will increase the targeting efficiency of cancer cells scattered in the tissue, providing a better protection from tumor recurrence. Here, the inventors demonstrate that AAV2 diffuses in brain parenchyma when injected directly in the organ and is able to infect the GBM CSCs with high efficiency, in vitro.

### Example 2

The inventors further demonstrated their detargeting approach using epigenetic silencer factor (ESF) transgenes. ESFs are engineered oncogenic and cancer-associated transcription factors that function as epigenetic repressors.

ESFs should not have any appreciable and harmful effect on heathy brain cells, which do not express the oncogenes of interest and are not proliferative cells. In such healthy cells, ESF induced decrease of some key cell-cycle genes may be detrimental to the cells. However, it is possible that ESF-dependent chromatin changes could alter neuronal performance in vivo over longer periods of time, e.g., if used for treatment in humans. Thus, the inventors have devised applied their detargeting strategy to restrict ESF expression to cancer cells after viral inoculation of the brain. To this end, a cassette was generated in which the 3′-UTR downstream to the transgene, e.g., the ESF cDNA, comprised a cluster of 4×TS for each of miRNA-124, -338-3p and -31:

CGATAAGCTTTGCAAAGATGGATAAAGTTTTAAACAGAGAGGAATCTTTGCAGCTAATGGAC
CTTCTAGGTCTTGAAAGGAGTGGGAATTGGCTCCGGTGCCCGTCAGTGGGCAGAGCGCACAT
CGCCCACAGTCCCCGAGAAGTTGGGGGGAGGGGTCGGCAATTGAACCGGTGCCTAGAGAAGG
TGGCGCGGGGTAAACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCAACGGGTTTGCCG
CCAGAACACAGGTAAGTGCCGTGTGTGGTTCCCGCGGGCCTGGCCTCTTTACGGGTTATGGC
CCTTGCGTGCCTTGAATTACTTCCACTGGCTGCAGTACGTGATTCTTGATCCCGAGCTTCGG
GTTGGAAGTGGGTGGGAGAGTTCGAGGCCTTGCGCTTAAGGAGCCCCTTCGCCTCGTGCTTG
AGTTGAGGCCTGGCCTGGGCGCTGGGGCCGCCGCGTGCGAATCTGGTGGCACCTTCGCGCCT
GTCTCGCTGCTTTCGATAAGTCTCTAGCCATTTAAAATTTTTGATGACCTGCTGCGACGCTT
TTTTTCTGGCAAGATAGTCTTGTAAATGCGGGCCAAGATCTGCACACTGGTATTTCGGTTTT
TGGGGCCGCGGGCGGCGACGGGGCCCGTGCGTCCCAGCGCACATGTTCGGCGAGGCGGGGCC
TGCGAGCGCGGCCACCGAGAATCGGACGGGGGTAGTCTCAAGCTGGCCGGCCTGCTCTGGTG
CCTGGCCTCGCGCCGCCGTGTATCGCCCCGCCCTGGGCGGCAAGGCTGGCCCGGTCGGCACC
AGTTGCGTGAGCGGAAAGATGGCCGCTTCCCGGCCCTGCTGCAGGGAGCTCAAAATGGAGGA
CGCGGCGCTCGGGAGAGCGGGCGGGTGAGTCACCCACACAAAGGAAAAGGGCCTTTCCGTCC
TCAGCCGTCGCTTCATGTGACTCCACGGAGTACCGGGCGCCGTCCAGGCACCTCGATTAGTT
CTCGAGCTTTTGGAGTACGTCGTCTTTAGGTTGGGGGGAGGGGTTTTATGCGATGGAGTTTC
CCCACACTGAGTGGGTGGAGACTGAAGTTAGGCCAGCTTGGCACTTGATGTAATTCTCCTTG
GAATTTGCCCTTTTTGAGTTTGGATCTTGGTTCATTCTCAAGCCTCAGACAGTGGTTCAAAG
TTTTTTTCTTCCATTTCAGGTGTCGTGAGGAATTCTGCAGTCGATCGACGGTACCGCGGGCC
CAACGGATCCACCATGGATGCTAAGTCACTGACTGCCTGGTCCCGGACACTGGTGACCTTCA
AGGATGTGTTTGTGGACTTCACCAGGGAGGAGTGGAAGCTGCTGGACACTGCTCAGCAGATC
CTGTACAGAAATGTGATGCTGGAGAACTATAAGAACCTGGTTTCCTTGGGTTATCAGCTTAC
TAAGCCAGATGTGATCCTCCGGTTGGAGAAGGGAGAAGAGCCCTGGCTGGTGGAGAGAGAAA
TTCACCAAGAGACCCATCCTGATTCAGAGACTGCATTTGAAATCAAATCATCAGTTGGCAGC
GGCGCATGCatgtacaacatgatggagacggagctgaagccgccgggcccgcagcaaacttc
ggggggcggcggcggcaactccaccgcggcggcggccggcggcaaccagaaaaacagcccgg
accgcgtcaagcggcccatgaatgccttcatggtgtggtcccgcgggcagcggcgcaagatg
gcccaggagaaccccaagatgcacaactcggagatcagcaagcgcctgggcgccgagtggaa
acttttgtcggagacggagaagcggccgttcatcgacgaggctaagcggctgcgagcgctgc
acatgaaggagcacccggattataaataccggccccggcggaaaaccaagacgctcatgaag
aaggataagtacacgctgcccggcgggctgctggcccccggcggcaatagcatggcgagcgg
ggtcggggtgggcgccggcctgggcgcgggcgtgaaccagcgcatggacagttacgcgcaca
tgaacggctggagcaacggcagctacagcatgatgcaggaccagctgggcGCTAGCGGCAGC

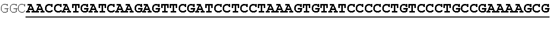

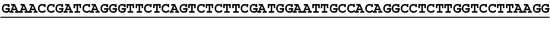

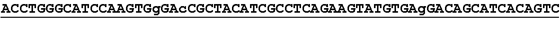

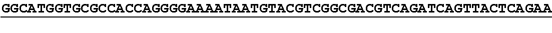

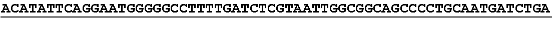

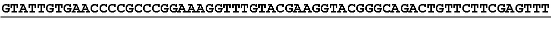

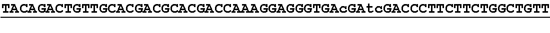

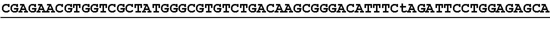

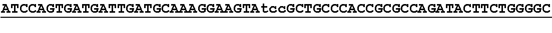

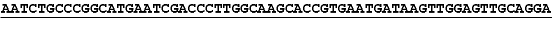

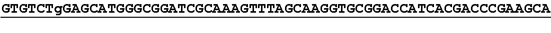

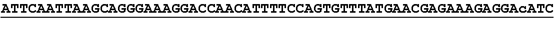

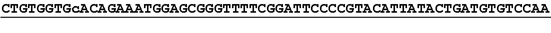

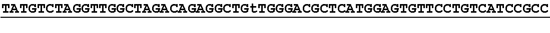

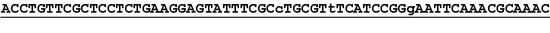

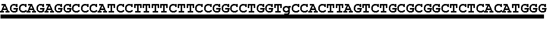

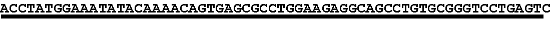

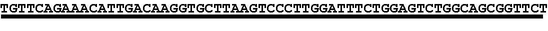

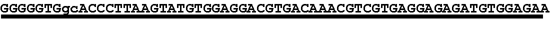

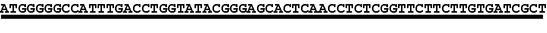

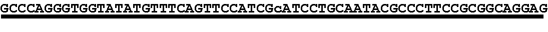

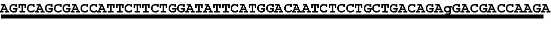

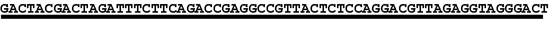

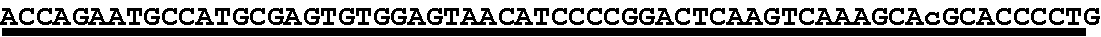

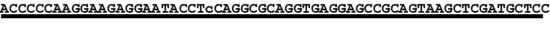

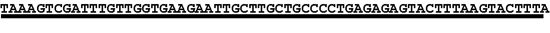

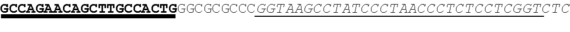

GATTCTACGTGAattaattaagaattcgacccagctttcttgtacaaagtggttgatatcca
gcacagtggcggccgctcgagtctagagggcccgcggttcgaaggtaagcctatccctaacc
ctctcctcggtctcgattctacgcgtaccggtgtcgactctagataaagcatcaagcttatc
gcgataccgtcgacaatcaacctctggattacaaaatttgtgaaagattgactggtattctt
aactatgttgctccttttacgctatgtggatacgctgctttaatgcctttgtatcatgctat
tgcttcccgtatggctttcattttctcctccttgtataaatcctggttgctgtctctttatg
aggagttgtggcccgttgtcaggcaacgtggcgtggtgtgcactgtgtttgctgacgcaacc
cccactggttggggcattgccaccacctgtcagctcctttccgggactttcgctttccccct
ccctattgccacggcggaactcatcgccgcctgccttgcccgctgctggacaggggctcggc
tgttgggcactgacaattccgtggtgttgtcggggaagctgacgtcctttccatggctgctc
gcctgtgttgccacctggattctgcgcgggacgtccttctgctacgtcccttcggccctcaa
tccagcggaccttccttcccgcggcctgctgccggctctgcggcctcttccgcgtcttcgcc
ttcgccctcagacgagtcggatctccctttgggccgcctccccgcctggaatgatatcctcg
agcccgggcgatatattgccttatttccgattattgccttatttcgcattattgccttattt

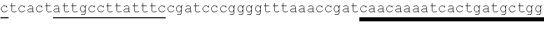

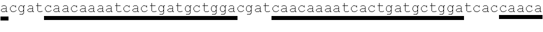

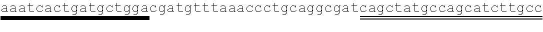

cgatcagctatgccagcatcttgcccgatcagctatgccagcatcttgcctcaccagctatg
ccagcatcttgcccgatcctgcaggagatct

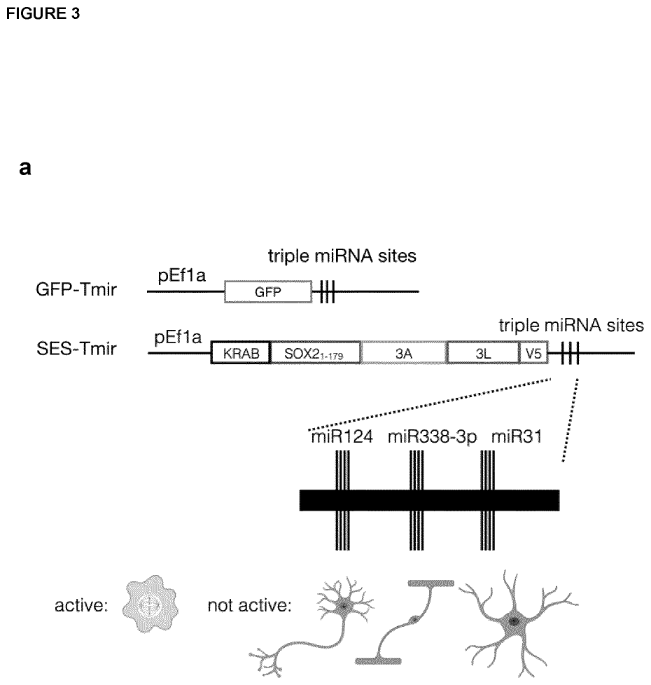

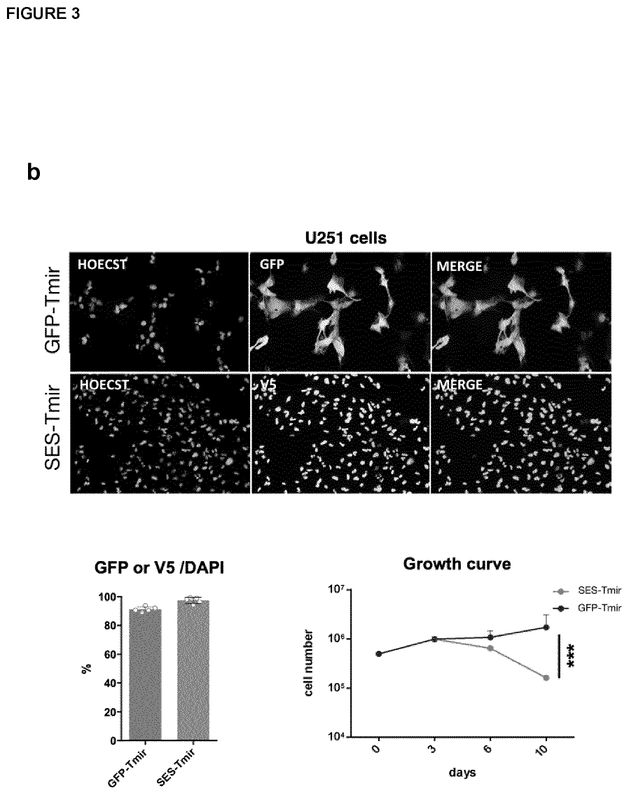

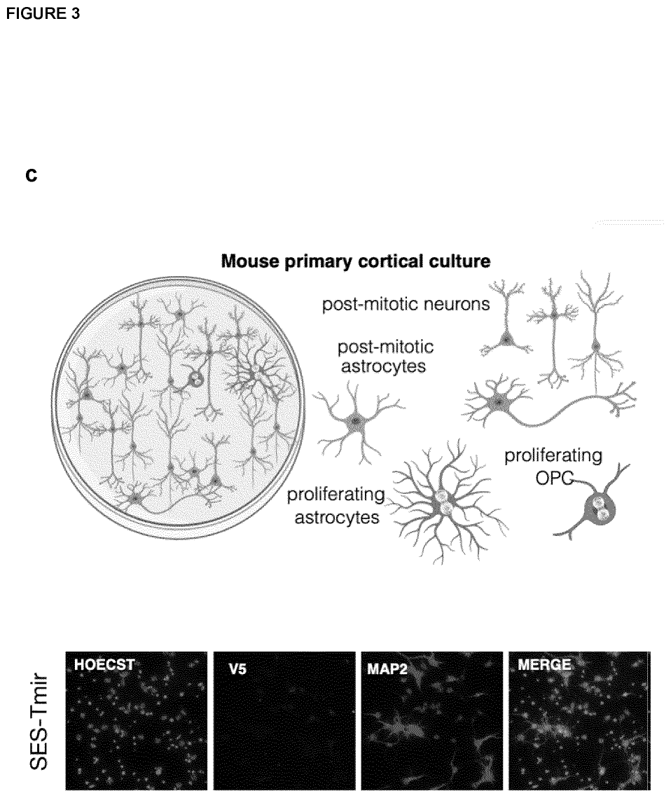

Thus, exogenous transgene expression, e.g., SES (an example ESF) expression as demonstrated in FIG. 3, should be silenced in all of the foregoing cell types through miRNA-dependent post-transcriptional silencing and block of protein translation (FIG. 3a). Notably, miRNA-124, -338-3p and -31, are not expressed in GBM, as demonstrated by GFP-Tmir and SES-Tmir lentiviruses (LVs), which exhibited unaffected transgene expression in cancer cells (FIG. 3b). The data herein further demonstrate that the presence of Tmir did not jeopardize SES activity, as the construct induced a significant cell loss in the transduced cancer cells in vitro (FIG. 3b, bottom right). The specificity of the miRNA detargeting system is demonstrated in FIG. 3c wherein the data show that, when used in primary cortical culture from mouse, no cells expressed the SES, as intended and due to the miRNA detargeting (FIG. 3c).

Detargeting the expression of the exogenous ESF from healthy brain cells, e.g., neurons, astrocytes and oligodendrocytes, by using miRNA target sequences within the 3′ UTR was demonstrated, thus affording improved safety and specificity. By utilizing target sequences of miRNAs that are highly expressed in brain cells, but not in tumor cells, the inventors provide polynucleotides and transgene expression cassettes with high specificity, e.g., for use as an anti-GBM treatment.

1. A polynucleotide comprising at least one miR-124 target sequence, and/or at least one miR-338-3p target sequence, and/or at least one miR-31 target sequence, wherein the miRNA target sequences are operably linked to a transgene.

2. The polynucleotide of claim 1, wherein the number of copies of each of the miRNA target sequences is independently selected from the group consisting of: one, two, three, and four.

3. The polynucleotide of claim 1 or claim 2, wherein the polynucleotide comprises four miR-124 target sequences, four miR-338-3p target sequences and four miR-31 target sequences, wherein the miRNA target sequences are operably linked to the transgene.

4. The polynucleotide of any one of claims 1 to 3, wherein:
(a) the miR-124 target sequence comprises or consists of a nucleotide sequence that has at least 90% sequence identity to SEQ ID NO: 1;
(b) the miR-338-3p target sequence comprises or consists of a nucleotide sequence that has at least 90% sequence identity to SEQ ID NO: 2; and/or
(c) the miR-31 target sequence comprises or consists of a nucleotide sequence that has at least 90% sequence identity to SEQ ID NO: 3.

5. The polynucleotide of any one of claims 1 to 4, wherein the miRNA target sequences are located after the transgene in the 5′ to 3′ direction.

6. The polynucleotide of any one of claims 1 to 5, wherein the miRNA target sequences or clusters of copies of the miRNA target sequences are, from 5′ to 3′, arranged in the order: miR-124 target sequence(s), miR-338-3p target sequence(s), and miR-31 target sequence(s).

7. The polynucleotide of any one of claims 1 to 6, wherein the miRNA target sequences are separated by spacer sequences.

8. The polynucleotide of any one of claims 1 to 7, wherein the polynucleotide comprises a nucleotide sequence that has at least 90% sequence identity to SEQ ID NO: 4.

9. The polynucleotide of any one of claims 1 to 8, wherein the polynucleotide further comprises a promoter operably linked to the transgene, optionally wherein the promoter is a tissue-specific promoter or a constitutive promoter, optionally a cancer cell-specific promoter or a proliferating cell-specific promoter.

10. The polynucleotide of claim 9, wherein the promoter is an Ef1a promoter or a Mki67 promoter.

11. A vector comprising the polynucleotide of any one of claims 1 to 10, optionally wherein the vector is a viral vector, optionally wherein the vector is a lentiviral vector or adeno-associated viral (AAV) vector.

12. A nanoparticle comprising the polynucleotide of any one of claims 1 to 10, or the vector of claim 11.

13. A cell comprising the polynucleotide of any one of claims 1 to 10, the vector of claim 11, or the nanoparticle of claim 12.

14. A composition comprising the polynucleotide of any one of claims 1 to 10, the vector of claim 11, the nanoparticle of claim 12, or the cell of claim 13.

15. The polynucleotide of any one of claims 1 to 10, the vector of claim 11, the nanoparticle of claim 12, the cell of claim 13, or the composition of claim 14 for use in therapy.

16. The polynucleotide of any one of claims 1 to 10, the vector of claim 11, the nanoparticle of claim 12, the cell of claim 13, or the composition of claim 14 for use in the treatment of cancer.
